(19) United States
(12) Patent Application Publication
(10) Pub. No.: US 2021/0340578 A1
(43) Pub. Date: Nov. 4, 2021

(54) ACID BISULFITE PRETREATMENT

(71) Applicant: Iogen Corporation, Ottawa (CA)

(72) Inventors: Brian FOODY, Ottawa (CA); Jeffrey S. TOLAN, Ottawa (CA)

(21) Appl. No.: 17/271,392

(22) PCT Filed: Nov. 9, 2018
(86) PCT No.: PCT/CA2018/000215

§ 371 Date: Feb. 25, 2021

Related U.S. Application Data
(60) Provisional application No. 62/725,583, filed on Aug. 31, 2018.

(51) Int. Cl. C12P 7/10; C12P 19/02

(52) U.S. Cl. CPC C12P 7/10; C12P 19/02

(57) ABSTRACT

A process for processing lignocellulosic biomass that includes pretreating lignocellulosic biomass, wherein the lignocellulosic biomass is heated in a pretreatment liquor containing sulfur dioxide and bisulfite salt, at a temperature between 120° C. and 150° C., for at least 30 minutes. The pH of the pretreatment liquor at 25° C. is less than 1.3, the concentration of sulfur dioxide is greater than 9.4 wt % (on liquor), and the concentration of alkali is between 0 wt % and 0.42 wt % (expressed as hydroxide, on liquor).

### CROSS-REFERENCE TO RELATED APPLICATIONS

This application claims the priority benefit of provisional application No. 62/725,583 filed Aug. 31, 2018, which is incorporated herein by reference.

### TECHNICAL FIELD

The present disclosure relates generally to a process and/or system for processing lignocellulosic biomass, and in particular, to a process and/or system for converting lignocellulosic biomass to glucose or an alcohol, where the lignocellulosic biomass is subject to a pretreatment with bisulfite prior to enzymatic hydrolysis.

### BACKGROUND

Lignocellulosic biomass refers to plant biomass that includes cellulose, hemicellulose, and lignin. Lignocellulosic biomass may be used to produce biofuels (e.g., ethanol, butanol, methane) by breaking down cellulose and/or hemicellulose into their corresponding monomers (e.g., sugars), which can then be converted to the biofuel via microorganisms. For example, glucose can be fermented to produce an alcohol such as ethanol or butanol.

While lignocellulosic biomass can be broken down into sugars solely using various chemical processes (e.g., acid hydrolysis), enzymatic hydrolysis is often the preferred approach for generating glucose as it is associated with higher yields, higher selectivity, lower energy costs, and/or milder operating conditions. For example, cellulose in lignocellulosic biomass may be converted to glucose by cellulases. However, as a result of the complicated structure of the plant cell wall, the enzymatic digestibility of cellulose in native lignocellulosic biomass is often low unless a large excess of enzyme is used (e.g., lignocellulosic biomass may be considered recalcitrant to biodegradation). Unfortunately, the cost of suitable enzymes can be high, and can significantly contribute to the overall costs of the process. Accordingly, it is advantageous for enzymatic hydrolysis to be preceded by a pretreatment process that makes the lignocellulosic biomass more amenable to enzymatic hydrolysis and/or reduces the amount of enzyme required.

Some examples of pretreatment processes that have been proposed for preparing lignocellulosic biomass for enzymatic hydrolysis include physical pretreatment (e.g., milling and grinding), dilute acid pretreatment, alkali pretreatment (e.g., lime), ammonia fiber expansion, hot water extraction, steam explosion, organic solvent, and/or wet oxidation.

It has been also proposed to prepare the lignocellulosic biomass with a pretreatment based on modified sulfite pulping. In sulfite pulping, various salts of sulfurous acid (H₂SO₃) are used to extract lignin from wood chips. The salts may be bisulfites (HSO₃⁻) and/or sulfites (SO₃²⁻), with sodium (Na⁺), calcium (Ca²⁺), potassium (K⁺), magnesium (Mg²⁺), or ammonium (NH₄⁻) counter ions. For example, the cooking liquor for a sulfite pulping process may be prepared by bubbling sulfur dioxide (SO₂) into a MgO solution. Sulfite pulping may be conducted in large pressure vessels call digesters, at temperatures between 130° C.-160° C., for 4-14 hours, depending on the chemicals used.

Sulfite pulping may be categorized as: (a) acid sulfite (e.g., pH 1-2); (b) bisulfite (e.g., pH 2-6); (c) neutral sulfite (e.g., pH 6-94); or (d) alkaline sulfite (e.g., pH 104) pulping. The composition of acid and bisulfite cooking liquor has been described using the total SO₂ content (e.g., SO₂ present as SO₂, H₂SO₃, HSO₃⁻, and/or SO₃²⁻) and/or combined SO₂ content (e.g., amount of SO₂ needed to produce XSO₃, where X is the counter ion). Acid sulfite cooking liquor has a high free SO₂ content compared to bisulfite cooking liquors (e.g., the free SO₂ and the combined SO₂ contents are substantially equal in bisulfite cooks).

Pretreatments based on acid sulfite pulping have been proposed. In general, such processes involve providing a certain level of bisulfite salt. For example, with regard to the Sulfite Pretreatment to Overcome Recalcitrance of Lignocellulose (SPORL) process, the addition of sulfite as a weak base has been stated to elevate the pH value of pretreatment liquor, which prevents hemicellulose and cellulose from excessive acid-catalyzed hydrolysis and subsequent decomposition to fermentation inhibitors (e.g., furfural and hydroxymethylfurfural (HMF)). In addition, with regard to SPORL, cellulose conversion has been found to be greater with increased bisulfite charge (e.g., in a H₂SO₄/NaHSO₃ system).

### SUMMARY

According to one aspect of the invention there is provided a process for processing lignocellulosic biomass comprising: (i) pretreating lignocellulosic biomass, said pretreating comprising heating the lignocellulosic biomass in a pretreatment liquor containing sulfur dioxide and bisulfite salt, said heating conducted between 120° C. and 150° C., for at least 30 minutes, wherein initially a pH of the pretreatment liquor at 25° C. is less than 1.3, a concentration of sulfur dioxide is greater than 9.4 wt %/o (on liquor), and a concentration of alkali is between 0 wt % and 0.42 wt % (expressed as hydroxide, on liquor); (ii) obtaining a slurry of pretreated lignocellulosic biomass produced in (i), said slurry having a solid fraction comprising cellulose and a liquid fraction comprising solubilized hemicellulose; (iii) forcing sulfur dioxide out of the liquid fraction, wherein said liquid fraction has a pH at 25° C. that is less than 1; (iv) enzymatically hydrolyzing at least a portion of the cellulose in the solid fraction to glucose; (v) fermenting the glucose to an alcohol, and (vi) recovering the alcohol.

According to one aspect of the invention there is provided a process for processing lignocellulosic biomass comprising: (i) pretreating lignocellulosic biomass, said pretreating comprising heating the lignocellulosic biomass in a pretreatment liquor containing sulfur dioxide and bisulfite salt, said heating conducted between 110° C. and 150° C., for at least 30 minutes, wherein initially a pH of the pretreatment liquor at 25° C. is less than 1.3, a concentration of sulfur dioxide is greater than 36 wt % (on dry solids), and a concentration of alkali is less than 0.25 wt/o (expressed as hydroxide, on liquor); (ii) obtaining a slurry of pretreated lignocellulosic biomass produced in (i), said slurry having a solid fraction comprising cellulose and a liquid fraction comprising solubilized hemicellulose; (iii) forcing sulfur dioxide out of the liquid fraction, wherein said liquid fraction has a pH at 25° C. that is less than 1; (iv) enzymatically hydrolyzing at least a portion of the cellulose in the solid fraction to glucose; (v) fermenting the glucose to an alcohol, and (vi) recovering the alcohol.

According to one aspect of the invention there is provided a process for processing lignocellulosic biomass comprising: (i) pretreating lignocellulosic biomass, said pretreating comprising heating the lignocellulosic biomass in a pretreatment liquor containing sulfur dioxide and bisulfite salt, said heating conducted between 110° C. and 150° C., for at least 30 minutes, wherein initially a pH of the pretreatment liquor at 25° C. is less than 1.3, and wherein a ratio of a concentration of sulfur dioxide on liquor to a concentration of alkali expressed as hydroxide, on liquor, is greater than 30; (ii) obtaining a slurry of pretreated lignocellulosic biomass produced in (i), said slurry having a solid fraction comprising cellulose and a liquid fraction comprising solubilized hemicellulose; (iii) forcing sulfur dioxide out of the liquid fraction, wherein said liquid fraction has a pH at 25° C. that is less than 1; (iv) enzymatically hydrolyzing at least a portion of the cellulose in the solid fraction to glucose; (v) fermenting the glucose to an alcohol, and (vi) recovering the alcohol.

### BRIEF DESCRIPTION OF THE DRAWINGS

FIG. 1 is a plot of glucose conversion versus time for the enzymatic hydrolysis of washed solids obtained from an acid bisulfite pretreatment according to one embodiment of the invention.

### DETAILED DESCRIPTION

Certain exemplary embodiments of the invention now will be described in more detail, with reference to the drawings, in which like features are identified by like reference numerals. The invention may, however, be embodied in many different forms and should not be construed as limited to the embodiments set forth herein.

The terminology used herein is for the purpose of describing certain embodiments only and is not intended to be limiting of the invention. For example, as used herein, the singular forms “a”, “an,” and “the” may include plural references unless the context clearly dictates otherwise. The terms “comprises”, “comprising”, “including”, and/or “includes”, as used herein, are intended to mean “including but not limited to”. The term “and/or”, as used herein, is intended to refer to either or both of the elements so conjoined. The phrase “at least one” in reference to a list of one or more elements, is intended to refer to at least one element selected from any one or more of the elements in the list of elements, but not necessarily including at least one of each and every element specifically listed within the list of elements. Thus, as a non-limiting example, the phrase “at least one of A and B” may refer to at least one A with no B present, at least one B with no A present, or at least one A and at least one B in combination. In the context of describing the combining of components by the “addition” or “adding” of one component to another, those skilled in the art will understand that the order of addition is not critical (unless stated otherwise). The terms “first”, “second”, etc., may be used to distinguish one element from another, and these elements should not be limited by these terms. Unless defined otherwise, all technical and scientific terms used herein have the same meanings as commonly understood by one of ordinary skill in the art.

The instant disclosure describes an improved pretreatment for lignocellulosic biomass that combines the use of a relatively low level of bisulfite salt with a relatively high SO₂ loading. As the pretreatment is conducted in the presence of bisulfite salt and SO₂, at low pH values (i.e., below 2), it may be referred to as an acid bisulfite pretreatment.

In general, the use of large amounts of SO₂ has been previously avoided in sulfite pulping and/or sulfite-based pretreatments because the chemical is expensive. For example, sulfite pulping liquors may contain less than about 10% total SO₂, by weight. Sulfite pretreatment liquors may contain even less. For example, in SPORL processes, a targeted total SO₂ concentration of 80 g/L (about 8 wt % by liquor) may be considered a high SO₂ loading.

In general, the presence of bisulfite salt may be considered beneficial for acid sulfite pulping and/or acid sulfite-based pretreatments as it is believed to promote lignin dissolution.

In addition, in SPORL, cellulose conversion has been found to increase with increasing bisulfite charge (e.g., in H₂SO₄/NaHSO₃ system).

In accordance with one embodiment of the invention, lignocellulosic biomass is subject to an acid bisulfite pretreatment that includes heating the lignocellulosic biomass at a temperature(s) between about 110° C. and about 160° C., for more than 30 minutes, in the presence of SO₂ and a bisulfite salt, where the concentration of SO₂ in the liquor is greater than 8 wt % (expressed as weight percent SO₂, based on weight of the pretreatment liquor), and wherein the concentration of alkali present and able to form the bisulfite salt is greater than 0 and less than about 0.42 wt % (expressed as weight percent OH, based on weight of the pretreatment liquor).

In accordance with one embodiment of the invention, lignocellulosic biomass is subject to an acid bisulfite pretreatment that includes heating the lignocellulosic biomass at a temperature(s) between about 110° C. and about 160° C., for more than 30 minutes, in the presence of SO₂ and a bisulfite salt, where the concentration of SO₂ in the liquor is between about 9.4 wt % and about 19.5 wt % (expressed as weight percent SO₂, based on weight of the pretreatment liquor), and wherein the concentration of alkali present and able to form the bisulfite salt is greater than 0 and less than about 0.42 wt % (expressed as weight percent OH, based on weight of the pretreatment liquor).

In accordance with one embodiment of the invention, lignocellulosic biomass is subject to an acid bisulfite pretreatment that includes heating the lignocellulosic biomass at a temperature(s) between about 110° C. and about 160° C., for more than 30 minutes, in the presence of SO₂ and a bisulfite salt, where the concentration of SO₂ is greater than about 36 wt % (based on dry solids), and wherein the concentration of alkali present and able to form the bisulfite salt is less than 0.25 wt % (expressed as weight percent OH, based on weight of the pretreatment liquor).

In accordance with one embodiment of the invention, lignocellulosic biomass is optionally subject to one or more preparatory steps, is subject to an acid bisulfite pretreatment, is hydrolyzed with enzymes, and is fermented to an alcohol. In one embodiment, excess SO₂ not consumed in the acid bisulfite pretreatment is recovered and/or recycled in the process.

### Lignocellulosic Biomass

In general, the lignocellulosic biomass may include and/or be derived from any lignocellulosic feedstock that may be pretreated in order to improve enzymatic digestibility. Lignocellulosic biomass refers to plant biomass that includes cellulose, hemicellulose, and lignin. The cellulose and hemicellulose fractions may be considered carbohydrate polymers, whereas lignin may be considered an aromatic polymer. Hydrolysis of the hemicellulose fraction may yield xylose, arabinose, mannose, galactose, and/or glucose, whereas hydrolysis of the cellulose fraction typically yields glucose. Since the cellulose, hemicellulose, and/or lignin fractions may be intertwined (e.g., cross-linked) hydrolysis of cellulose in the lignocellulosic biomass may be difficult without a pretreatment step.

In one embodiment, the lignocellulosic biomass has a combined content of cellulose, hemicellulose, and lignin that is greater than about 25 wt %, that is greater than about 50 wt %, or is greater than about 75 wt %. In one embodiment, sucrose, fructose, and/or starch are also present, but in lesser amounts than cellulose and hemicellulose.

In one embodiment, the lignocellulosic biomass is a lignocellulosic feedstock selected from: (i) energy crops; (ii) residues, byproducts, or waste from the processing of plant biomass in a facility or feedstock derived therefrom; (iii) agricultural residues; (iv) forestry biomass; (v) waste material derived from pulp and paper products; (vi) pulp and paper waste; and/or (vii) municipal waste including components removed from municipal waste.

Energy crops include biomass crops such as grasses, including C4 grasses, such as switch grass, energy cane, sorghum, cord grass, rye grass, miscanthus, reed canary grass, C3 grasses such as Arundo donax, or a combination thereof.

Residues, byproducts, or waste from the processing of plant biomass include residues remaining after obtaining sugar from plant biomass (e.g., sugar cane bagasse, sugar cane tops and leaves, beet pulp, Jerusalem artichoke residue), and residues remaining after grain processing (e.g., corn fiber, corn stover, and bran from grains). Agricultural residues include, but are not limited to soybean stover, corn stover, sorghum stover, rice straw, sugar cane tops and/or leaves, rice hulls, barley straw, wheat straw, canola straw, oat straw, oat hulls, corn fiber, and corn cobs.

Forestry biomass includes hardwood, softwood, recycled wood pulp fiber, sawdust, trimmings, and/or slash from logging operations. Pulp and paper waste includes waste from chemical pulping such as black liquor, spent sulfite liquor, sludge, and/or fines.

Municipal waste includes post-consumer material or waste from a variety of sources such as domestic, commercial, institutional and/or industrial sources.

In one embodiment, the lignocellulosic biomass is an energy crop or biomass crop. In one embodiment, the lignocellulosic biomass comprises an agricultural residue. In one embodiment, the lignocellulosic biomass comprises a non-woody lignocellulosic feedstock. In one embodiment, the lignocellulosic biomass comprises hardwood. In one embodiment, the lignocellulosic biomass comprises softwood. In one embodiment, the lignocellulosic biomass comprises wheat straw, or another straw. In one embodiment, the lignocellulosic biomass comprises stover. The term “straw” may refer to the stem, stalk and/or foliage portion of crops remaining after the removal of starch and/or sugar containing components for consumption. Examples of straw include, but are not limited to sugar cane tops and/or leaves, bagasse, oat straw, wheat straw, rye straw, rice straw and barley straw. The term “stover” may include the stalk and foliage portion of crops after the removal of starch and/or sugar containing components of plant material for consumption. Examples of stover include, but are not limited to, soybean stover, sorghum stover, and corn stover. In one embodiment, the lignocellulosic biomass is a mixture of fibers that originate from different kinds of plant materials, including mixtures of cellulosic and non-cellulosic feedstock. In one embodiment, the lignocellulosic biomass is a second generation feedstock.

### Biomass Preparation

In general, the lignocellulosic biomass may be subjected to one or more optional preparatory steps prior to the pretreatment and/or as part of the pretreatment. Some examples of biomass preparation include size reduction, washing, leaching, sand removal, soaking, wetting, slurry formation, dewatering, plug formation, addition of heat, and addition of chemicals (e.g., pretreatment and/or other). In general, these preparatory steps may depend on the type of biomass and/or the selected pretreatment conditions.

In one embodiment, the lignocellulosic biomass is subjected to a size reduction. Some examples of size reduction methods include milling, grinding, agitation, shredding, compression/expansion, and/or other types of mechanical action. Size reduction by mechanical action may be performed by any type of equipment adapted for the purpose, for example, but not limited to, hammer mills, tub-grinders, roll presses, refiners, hydropulpers, and hydrapulpers. In one embodiment, lignocellulosic feedstock having an average particle size that is greater than about 6-8 inches is subject to a size reduction wherein at least 90% by volume of the particles produced from the size reduction have a length between about 1/16 inch and about 6 inches.

In one embodiment, the lignocellulosic biomass is washed and/or leached with a liquid (e.g., water and/or an aqueous solution). Washing, which may be performed before, during, or after size reduction, may remove sand, grit, fine particles of the lignocellulosic biomass, and/or other foreign particles that otherwise may cause damage to the downstream equipment. Leaching, which may be performed before, during, or after size reduction, may remove soluble components from the lignocellulosic biomass. For example, leaching may remove salts and/or buffering agents.

In one embodiment, the lignocellulosic biomass is subject to sand removal. For example, in one embodiment, the lignocellulosic biomass is washed to remove sand. Alternatively, or additionally, sand may be removed using other wet or dry sand removal techniques that are known in the art (e.g., including the use of a hydrocyclone or a sieve).

In one embodiment, the lignocellulosic biomass is soaked in water and/or an aqueous solution (e.g., comprising a pretreatment chemical). Soaking the lignocellulosic biomass may allow pretreatment chemical(s) to more uniformly impregnate the biomass, which in turn may provide even cooking in the heating step of pretreatment. For example, soaking the biomass in a solution comprising a pretreatment chemical may provide uniform impregnation of the pretreatment chemical. In general, soaking may be carried out at any suitable temperature and/or for any suitable duration.

In one embodiment, the lignocellulosic biomass is slurried in liquid (e.g., water), which allows the lignocellulosic biomass to be pumped. In one embodiment, the lignocellulosic biomass is slurried subsequent to size reduction, washing, and/or leaching. The desired weight ratio of water to dry biomass solids in the slurry may be determined by factors such as pumpability, pipe-line requirements, and other practical considerations. In general, slurries having a consistency less than about 10 wt % may be pumped using a relatively inexpensive slurry pump.

In one embodiment, the lignocellulosic biomass is at least partially dewatered (e.g., at least some water is removed). In one embodiment, the lignocellulosic biomass is at least partially dewatered to provide a specific consistency.

In one embodiment, the lignocellulosic biomass is at least partially dewatered in order to increase the undissolved solids content relative to the incoming biomass. In one embodiment, the lignocellulosic biomass is at least partially dewatered in order to remove at least some of the liquid introduced during washing, leaching, slurrying, and/or soaking. In one embodiment, dewatering is achieved using a drainer, filtration device, screen, screw press, and/or extruder. In one embodiment, dewatering is achieved using a centrifuge. In one embodiment, the dewatering is achieved prior to and/or as part of plug formation. In general, plug formation may be considered an integration of lignocellulosic biomass particles into a compacted mass referred to herein as a plug. Plug formation devices may or may not form a plug that acts as a seal between areas of different pressure. Some examples of plug formation devices that dewater biomass include a plug screw feeder, a pressurized screw press, a co-axial piston screw feeder, and a modular screw device.

As mentioned above, each of the washing, leaching, slurrying, soaking, dewatering, and preheating stages are optional and may or may not be included in the process.

In general, each of these options may be associated with potential advantages and/or disadvantages. For example, some lignocellulosic feedstock may have a significant inorganic content (e.g., a relatively high K⁺, Na⁺, Ca²⁺, Mg²⁺ content). For example, energy crops and/or agricultural residues may contain significant amounts of potassium carbonate (K₂CO₃), calcium carbonate (CaCO₃), and/or sodium carbonate (Na₂CO₃). As these soluble salts may consume acid during an acid pretreatment, they may be referred to as alkali inherent to the biomass (e.g., inherent alkali). Washing or leaching the biomass may reduce or remove the amount of alkali inherent to the feedstock, and thus may provide a more consistent inherent alkali level, thereby improving the pretreatment by making the amount of acid required more consistent/predictable. However, by reducing or removing the amount of inherent alkali in the biomass, cations (e.g., K⁺, Na⁺, Ca²⁺, Mg²⁺) that could be useful in the acid bisulfite pretreatment are removed. In addition, washing or leaching the biomass may require additional water and processing equipment (e.g., washing equipment), each of which is an additional expense. Subjecting the lignocellulosic biomass to a water soaking step may be advantageous in that it can even out the inherent alkali concentration without removing a significant amount of K⁺, Na⁺, Ca²⁺, and/or Mg²⁺.

In one embodiment, washing, leaching, soaking and/or dewatering of the biomass is conducted at a temperature between about 20° C. and 90° C., for 2 to 20 minutes. In one embodiment, wash liquor is pooled in a volume sufficiently large to maintain a uniform inherent alkali concentration over a period of at least several minutes.

Pretreatment

The term “pretreating” or “pretreatment”, as used herein, refers to one or more steps wherein lignocellulosic biomass is treated to improve the enzymatic digestibility thereof. For example, in one embodiment, the pretreatment disrupts the structure of the lignocellulosic material such that the cellulose therein is more susceptible and/or accessible to enzymes in a subsequent enzymatic hydrolysis of the cellulose.

Without pretreatment, even when excess enzyme is added and the hydrolysis extends over multiple days, the maximum amount of glucose obtained from a feedstock such as wheat straw may be less than about 10-15 wt % (based on cellulose available in the feedstock). In one embodiment, the pretreatment conditions are selected to improve the enzymatic digestibility of the lignocellulosic feedstock, thereby increasing the glucose yield and/or increasing the rate of hydrolysis (for a given yield). In one embodiment, pretreating the lignocellulosic biomass allows at least 50 wt %, at least 60 wt %, at least 70 wt %, at least 80 wt %, or at least 90 wt % of the cellulose in the lignocellulosic biomass to be converted to glucose (based on the cellulose available in the biomass).

In one embodiment, the pretreatment conditions are selected to provide a relatively high glucose yield from the cellulose fraction, and a relatively high product yield from the hemicellulose fraction. Hemicelluloses include xylan, arabinoxylan, glucomannan, and galactans. Xylan may be the most common, and is mainly composed of xylose. A high xylose yield is advantageous because it is generally associated with a lower production of compounds that are potentially inhibitory to the hydrolysis and/or fermentation (e.g., xylose can degrade to furfural), and thus may be associated with a higher ethanol yield from the cellulose fraction. In addition, since xylose can be converted to ethanol or another product (e.g., xylitol), the overall product yield from the lignocellulosic biomass may be increased. In any case, a high xylose yield may indicate that much of the hemicellulose has been solubilized, which may improve the enzymatic digestibility of the cellulose. In one embodiment, pretreating the lignocellulosic biomass includes solubilizing at least about 50 wt %, at least about 60 wt %, at least about 70 wt %, at least about 80 wt %, or at least about 90 wt % of the xylan in the biomass.

In general, the pretreatment includes an acid bisulfite pretreatment. The acid bisulfite pretreatment includes heating the lignocellulosic biomass in the presence of sulfur dioxide (SO₂) and one or more bisulfite salts (HSO₃⁻ salts). The bisulfite salts, which for example may have Na⁺, Ca²⁺, K⁺, Mg²⁺, or NH₄⁺ counter ions, may be added directly (e.g., added as NaHSO₃) and/or may be formed in solution (e.g., by introducing the SO₂ into a solution containing alkali (e.g., a NaOH solution), or by adding sulfite salts to an aqueous SO₂ solution).

In general, the acid bisulfite pretreatment is conducted at a temperature between about 110° C. and about 160° C. In one embodiment, the acid bisulfite pretreatment is conducted at a temperature(s) between about 120° C. and about 150° C. In one embodiment, the acid bisulfite pretreatment is conducted at a temperature(s) between about 120° C. and about 145° C. In one embodiment, the acid bisulfite pretreatment is conducted at a temperature(s) between about 125° C. and about 140° C. Using pretreatment temperatures between about 110° C. and about 150° C. advantageously avoids the equipment and/or xylose degradation associated with pretreatments at relatively high temperatures (e.g., greater than 160° C.).

In general, the acid bisulfite pretreatment is conducted for at least 30 minutes. In one embodiment, the acid bisulfite pretreatment is conducted at a temperature(s) between about 120° C. and about 150° C. for at least 60 minutes, at least 80 minutes, at least 90 minutes, at least 100 minutes, at least 120 minutes, at least 140 minutes, at least 160 minutes, at least 180 minutes, at least 200 minutes, at least 220 minutes, or about 240 minutes. In one embodiment, the acid bisulfite pretreatment is conducted at a temperature(s) between about 120° C. and about 140° C. for at least 60 minutes, at least 80 minutes, at least 90 minutes, at least 100 minutes, at least 120 minutes, at least 140 minutes, at least 160 minutes, at least 180 minutes, at least 200 minutes, at least 220 minutes, or about 240 minutes. In one embodiment, the acid bisulfite pretreatment is conducted at a temperature(s) between about 120° C. and about 140° C. for a time between about 30 minutes and 240 minutes.

Using pretreatment temperatures between about 120° C. and about 140° C. for at least 60 minutes advantageously allows a significant amount of the lignin to become sulfonated. Using pretreatment temperatures between about 120° C. and about 140° C. for between 120 minutes and 240 minutes may promote significant xylan dissolution and significant lignin dissolution, without producing excessive degradation products. The pretreatment time does not include the time to warm up the pretreatment liquor and lignocellulosic biomass to at least 110° C.

In general, the acid bisulfite pretreatment includes adding SO₂. The SO₂ may be added as a gas, in an aqueous solution, or as a liquid (e.g., under pressure). When in an aqueous solution (e.g., dissolved in water), SO₂ also may be referred to as sulfurous acid (H₂SO₃). In one embodiment, the SO₂ is added to the biomass before entering the pretreatment reactor (e.g., in an acid soak). In one embodiment, the SO₂ is added to the biomass in the pretreatment reactor. In one embodiment, the SO₂ is added to the biomass before entering the pretreatment reactor and in the pretreatment reactor. In one embodiment, the SO₂ added includes freshly-added SO₂ (e.g., new to the process). In one embodiment, the SO₂ added includes recycled SO₂ (e.g., recovered from and/or reused within the process). In one embodiment, the SO₂ added includes make-up SO₂ (e.g., used to top up the amount of SO₂ present). In one embodiment, the SO₂ is added as a H₂SO₃ solution prepared by dissolving SO₂ in water. In one embodiment, the SO₂ is added as a HSO₃⁻ salt containing solution, which is prepared by dissolving SO₂ in an aqueous solution containing alkali. Optionally, one or more other acids (e.g., H₂SO₄ or HCl) are added.

In general, the SO₂ added to the pretreatment may be present as SO₂, H₂SO₃, HSO₃⁻, and/or SO₃²⁻, according to the following reactions:

SO₂+H₂O<=>H₂SO₃ (1)

H₂SO₃+H₂O<=>HSO₃⁻+H₃O⁺ (2)

HSO₃⁻+H₂O<=>SO₃²⁻+H₃O⁺ (3)

The “concentration of SO₂” or “SO₂ concentration”, takes into account contributions from H₂SO₃, HSO₃⁻, and SO₃²⁻, expressed on a molar-equivalent-to-SO₂ basis, but expressed as weight percent SO₂. However, at the conditions used in the acid bisulfite pretreatment (e.g., pH values less than about 1.3), the equilibrium in equation (3) will be shifted to the left and there will be negligible contributions from SO₃²⁻. The weight percent of SO₂ may be based on the total pretreatment liquor weight (on liquor), or based on the dry solids weight (on dry solids). The total pretreatment liquor weight includes the weight of moisture in the biomass, but not the weight of the dry solids.

In one embodiment, sufficient SO₂ is added to provide a SO₂ concentration at the start of pretreatment that is greater than about 9.4 wt/o (on liquor). In one embodiment, sufficient SO₂ is added to provide a SO₂ concentration at the start of pretreatment that is between about 9.4 wt % and about 19.5 wt % (on liquor). For reference, a SO₂ concentration between about 9.4 wt % and about 19.5 wt % (on liquor) is roughly equivalent to a H₂SO₃ concentration between about 12 wt % and about 25 wt % (on liquor). In one embodiment, sufficient SO₂ is added to provide a SO₂ concentration at the start of pretreatment that is greater than about 6 wt %, greater than about 7 wt %, greater than about 7.5 wt %, greater than about 8 wt %, greater than about 8.5 wt %, greater than about 9 wt %, greater than about 9.5 wt/o, or greater than about 10 wt % (i.e., on liquor).

The concentration of SO₂ based on dry solids may be determined using the consistency of the lignocellulosic biomass slurry. In general, the term consistency refers the amount of undissolved dry solids or “UDS” in a sample, and is often expressed as a ratio on a weight basis (wt:wt), or as a percent on a weight basis, for example, % (w/w), also denoted herein as wt %. For example, consistency may be determined by filtering and washing the sample to remove dissolved solids and then drying the sample at a temperature and for a period of time that is sufficient to remove water from the sample, but does not result in thermal degradation of the sample. The dry solids are weighed. The weight of water in the sample is the difference between the weight of the wet sample and the weight of the dry solids.

In one embodiment, the acid bisulfite pretreatment is conducted at a solids consistency between about 10 wt % and about 40 wt %. In one embodiment, the acid bisulfite pretreatment is conducted at a solids consistency between about 20 wt % and about 40 wt %. In one embodiment, the acid bisulfite pretreatment is conducted at a solids consistency between about 20 wt % and about 35 wt %. In one embodiment, the acid bisulfite pretreatment is conducted at a solids consistency between about 10 wt % and about 25 wt %. A SO₂ concentration that is between about 9.4 wt % and about 19.5 wt % (on liquor) corresponds to a SO₂ concentration that is between about 84.3 wt % and about 175.6 wt % (on dry solids) at a consistency of about 10 wt %, or between about 14.0 wt % and about 29.3 wt % (on dry solids) at a consistency of about 40 wt %, respectively. A consistency of about 10 wt % may correspond approximately to a liquid to solids ratio of about 9:1, whereas a consistency of about 40 wt % may correspond approximately to a liquid to solid ratio of about 1.5:1. In one embodiment, sufficient SO₂ is added to provide a SO₂ concentration at the start of pretreatment that is greater than about 35 wt %, greater than about 40 wt %, greater than about 45 wt %, greater than about 50 wt %, greater than about 55 wt %, greater than about 60 wt %, greater than about 65 wt %, greater than about 70 wt %, or greater than about 75 wt % (i.e., on dry solids). In one embodiment, sufficient SO₂ is added to provide a SO₂ concentration at the start of pretreatment that is greater than about 36 wt %.

In general, the concentration of SO₂ (on liquor, or dry solids) is determined using titration (e.g., with potassium iodate). However, as this may be challenging when relatively high SO₂ concentrations are achieved by introducing SO₂ into a pressurizable reactor, the concentration of SO₂ may be determined using the SO₂ loading. The “SO₂ loading” refers to the amount of SO₂ fed to the pretreatment per amount of dry lignocellulosic biomass fed to the system (e.g., as a weight percentage (wt %)). If the reactor has a large headspace (e.g., greater than 75% of the total reactor volume), the concentration of SO₂ can take into account the volume of the reactor headspace and partitioning of SO₂ into the vapour phase.

In general, bisulfite salts may be formed by reacting an alkali (base) with sulfurous acid, or by bubbling SO₂ into a solution containing alkali (base). For example, consider the following acid-base reaction:

H₂SO₃+MOH<=>MHSO₃+H₂O (4)

where M may be referred to as the counter cation. Some examples of alkali suitable for use in the acid bisulfite pretreatment include NaOH, NaHCO₃, Na₂CO₃, KOH, KHCO₃, K₂CO₃, CaCO₃, MgO, NH₃, etc.

As the alkali may be provided as a hydroxide, carbonate salt, or other form, for comparative purposes, the “concentration of alkali” or “alkali concentration” may be expressed on a molar-equivalent-to-M basis, where M is the cation on a monovalent basis (Na⁺, K⁺, NH₄⁺, ½Ca²⁺, ½Mg²⁺), but expressed as weight percent hydroxide (OH).

In one embodiment, the alkali concentration at the start of pretreatment is greater than about 0 wt % and less than about 0.42 wt % (OH, on liquor). An alkali concentration that is about 0.42 wt % (OH, on liquor) corresponds to an alkali concentration that is about 3.78 wt % (OH, on dry solids) for a consistency of about 10 wt %, or about 0.63 wt % (OH, on dry solids) for a consistency of about 40 wt %. For reference, if the alkali is only provided by adding NaOH, an alkali concentration of about 0.42 wt % (OH, on liquor) is roughly equivalent to a NaOH charge of about 0.99 wt %, which alternatively may correspond to a NaHSO₃ charge of about 2.56 wt % (on liquor). If the alkali is only provided by adding CaCO₃, an alkali concentration of about 0.42 wt % (OH, on liquor) is roughly equivalent to a CaCO₃ charge of about 1.24 wt % (on liquor) or a Ca(HSO₃)₂ charge of about 2.47 wt % (on liquor).

The alkali concentration refers to concentration of alkali present and able to form a bisulfite salt. Accordingly, the alkali concentration may include alkali inherent to the feedstock (e.g., K₂CO₃, CaCO₃, and/or Na₂CO₃) and/or alkali added for the pretreatment (e.g., NaOH, NaHSO₃ NaHCO₃, Na₂CO₃, Na₂SO₃ KOH, KHCO₃, K₂CO₃, CaCO₃, CaO, MgO, NH₃, etc.). For example, without adding alkali and without washing, wheat straw may have an inherent alkali concentration that is between about 0.15 wt % and about 0.63 wt % (OH, on dry solids), whereas bagasse may provide an inherent alkali concentration as high as about 0.2 wt % (OH, on dry solids). Woody feedstock tends to have a much lower, or even negligible, alkali concentration. The alkali concentration on the dry solids may be converted to the alkali on liquor by taking the solids consistency into account.

In one embodiment, the acid bisulfite pretreatment includes adding alkali. The alkali, which may be added as a solid or in an aqueous solution, may be added in any order (e.g., with regard to SO₂ and the lignocellulosic biomass). For example, the alkali may be added to water or an aqueous solution containing SO₂ in order to prepare an acid bisulfite liquor that is contacted with the lignocellulosic biomass. Alternatively, the lignocellulosic biomass may be contacted first with a solution containing alkali or SO₂, and then contacted with a solution containing the other of the alkali or SO₂.

In one embodiment the acid bisulfite liquor is prepared by treating a H₂SO₃ solution with alkali in an acid base reaction. In one embodiment, the alkali comprises an alkali or alkaline earth element (as the hydroxide or carbonate salt). In one embodiment, the acid bisulfite liquor is prepared by bubbling SO₂ into an aqueous solution containing the alkali (e.g., bubbling SO₂ into an aqueous solution prepared by adding MgO to water). In one embodiment, the acid bisulfite liquor is prepared by mixing a H₂SO₃ solution with a bisulfite salt solution. In one embodiment, the acid bisulfite pretreatment includes adding a source of counter cation such as K⁺, Na⁺, Ca²⁺, Mg²⁺, or NH⁴⁺ that readily forms a bisulfite salt. In one embodiment, the acid bisulfite pretreatment includes adding a bisulfite salt (e.g., NaHSO₃, KHSO₃, Ca(HSO₃)₂, Mg(HSO₃)₂). Adding a bisulfite salt is advantageous in that it can supply the system with both alkali and SO₂. In one embodiment, the acid bisulfite pretreatment includes adding a hydroxide or carbonate salt of K⁺, Na⁺, or NH₄⁺. Advantageously, hydroxides and/or carbonates based on these monovalent cations are generally more soluble than the hydroxides and/or carbonates based on divalent cations. In one embodiment, alkali is recovered from the process (e.g., in a preparatory leaching step, or from lignosulfonate produced by the process) and added to the pretreatment. In one embodiment, the alkali used in the acid bisulfite pretreatment is primarily extraneous. In one embodiment, the alkali used in the acid bisulfite pretreatment is a combination of extraneous alkali and alkali inherent to the lignocellulosic biomass. In one embodiment, the acid bisulfite pretreatment includes adding the lignocellulosic biomass to an aqueous solution having an alkali concentration that is greater than about 0 wt % and less than about 0.42 wt % (OH, based on liquor).

The concentration of alkali (on liquor, or dry solids), may be determined using the mass of alkali added to pretreatment and/or the mass of inherent alkali. For example, for lignocellulosic biomass that does not contain significant amounts of inherent alkali (e.g., pine), the concentration of alkali may be determined solely using the amount of alkali added to the pretreatment. For lignocellulosic biomass that contains significant amounts of inherent alkali, the alkali concentration may be determined using the amount of alkali added to the pretreatment, in addition to the amount of alkali inherent to the lignocellulosic biomass. The amount of alkali inherent to the lignocellulosic biomass may be determined by preparing a solution of sulfuric acid (H₂SO₄) in water at pH 1.05, 25° C., adding the feedstock to a weight of 5% (dry basis), measuring the resulting pH, and calculating from the acid-base equilibrium of H₂SO₄ the weight of OH as a percentage of the weight of feedstock.

In one embodiment, the acid bisulfite pretreatment includes adding alkali to SO₂ in a ratio that results in excess SO₂ (e.g., such that the pH is below about 2). In general, the pH of the pretreatment may be dependent upon the amount of SO₂ added and/or the amount of alkali available to form bisulfite salts. Pretreating with SO₂ and bisulfite salt is advantageous because it may sulfonate the lignin, thereby modifying the structure of the lignin, and/or may dissolve lignin and/or hemicellulose. In sulfonating lignin, lignosulfonic acid may be produced. Lignosulfonic acid is a strong acid that may promote hemicellulose dissolution. Since lignosulfonic acid is a stronger acid than SO₂, the pH of the slurry may drop as the pretreatment progresses (e.g., from some initial pH to some final pH).

In general, the acid bisulfite pretreatment is conducted at a pH below about 2. In one embodiment, sufficient SO₂ is added to provide an initial pH below about 1.3. In one embodiment, sufficient SO₂ is added to provide an initial pH below about 1.25. In one embodiment, sufficient SO₂ is added to provide an initial pH below about 1.2. In one embodiment, sufficient SO₂ is added to provide an initial pH below about 1.25. In one embodiment, sufficient SO₂ is added to provide an initial pH below about 1. In one embodiment, sufficient SO₂ is added to provide an initial pH between about 1.3 and about 0.4. In one embodiment, sufficient SO₂ is added to provide an initial pH between about 1.25 and about 0.7. The “initial pH” refers to the pH of the lignocellulosic biomass slurry, at ambient temperature, at the start of the pretreatment (e.g., after the SO₂ has been added, but before heating).

In one embodiment, sufficient SO₂ is added to provide a slurry of pretreated material (pretreated slurry) having a final pH less than about 1. In one embodiment, sufficient SO₂ is added to provide a pretreated slurry having a final pH less than about 0.9. In one embodiment, sufficient SO₂ is added to provide a pretreated slurry having a final pH less than about 0.8. In one embodiment, sufficient SO₂ is added to provide a pretreated slurry having a final pH less than about 0.7. In one embodiment, sufficient SO₂ is added to provide a pretreated slurry having a final pH less than about 0.6. In one embodiment, sufficient SO₂ is added to provide a pretreated slurry having a final pH between about 1 and about 0.3. The “final pH” refers to the pH of the pretreated slurry, at ambient temperature, at the end of the pretreatment (e.g., after the pretreated material is discharged from the pretreatment reactor(s)).

In general, the SO₂ concentration of a H₂SO₃ solution may be limited by the solubility of SO₂ in water. For example, if no alkali is added, the SO₂ concentration may be limited to below about 10 wt % (on liquor) at about 23° C. In one embodiment, a SO₂ concentration that is between about 9.4 wt % and about 19.5 wt % (on liquor) is obtained by bubbling in SO₂ into water or an aqueous alkali solution that is actively cooled. In one embodiment, a SO₂ concentration that is between about 9.4 wt/o and about 19.5 wt % (on liquor) is obtained by introducing the SO₂ under pressure. In one embodiment, SO₂ is introduced into a vessel to provide an SO₂ partial pressure of about 18 psia to about 37 psia, at 25° C. In any case, the pretreatment liquor may or may not be heated prior to entering the pretreatment reactor (e.g., heated under pressure). In one embodiment, the amount of SO₂ and/or alkali added is selected such that initially (e.g., near the start of pretreatment) the pH of the pretreatment liquor at 25° C. is less than 1.3, a concentration of sulfur dioxide is greater than 9.4 wt % (on liquor), and a concentration of alkali, expressed as hydroxide, is between 0 wt % and 0.42 wt % (on liquor).

Providing a limited amount of alkali while increasing the amount of SO₂ provided may have numerous advantages.

In one embodiment, sufficient SO₂ is added to provide a ratio of

$$\frac{SO 2 concentration ( on liquor )}{Alkali concentration ( OH , on liquor )} > 20 \qquad ( 5 )$$

For example, for an alkali concentration of 0.42 wt % (OH, on liquor), and a SO₂ concentration of 9.4 wt % (on liquor), the ratio is about 22. For an alkali concentration of 0.42 wt % (OH, on liquor), and a SO₂ concentration of 19.5 wt % (on pretreatment liquor), the ratio is about 46. In one embodiment, sufficient SO₂ is added such that the ratio is greater than 25, greater than 30, greater than 35, or greater than 40.

In one embodiment, sufficient SO₂ is added to provide a SO₂ concentration that is greater than about 36 wt/o (on dry solids), while the concentration of alkali is less than 0.25 wt % (OH, on liquor). In one embodiment, sufficient SO₂ is added to provide a SO₂ concentration that is greater than about 40 wt % (on dry solids), greater than about 45 wt/o (on dry solids), or greater than about 50 wt % (on dry solids), while the concentration of alkali is less than 0.25 wt % (OH, on liquor).

In one embodiment, the concentration of alkali is selected to be less than the concentration of organic acids produced in the pretreated slurry. More specifically the concentration of alkali expressed as moles cation per liter is less than the concentration of organic acid present after pretreatment, expressed as moles per liter. The organic acids present in the pretreated slurry may include acetic acid, glucuronic acid, and methyl glucuronic acid. For example, acetic acid may be formed by the hydrolysis of acetyl groups in hemicellulose. Other acids such as coumaric acid and ferulic acid, or gluconic acids arising from the degradation of glucose and xylose, may also be present. The concentration of organic acids is expressed as equivalent molar concentration of acetic acid.

Limiting the amount of alkali present during the pretreatment to a concentration less than about 0.42 wt % (OH, on liquor), while increasing the amount of SO₂ to a level that provides an initial pH less than 1.3, may have numerous advantages.

For example, limiting the amount of alkali present may significantly improve SO₂ recovery. In solution, SO₂ (as a dissolved gas) is in equilibrium with HSO₃⁻. This equilibrium is dependent upon the pH.

SO₂+H₂O<=>HSO₃⁻+H₃O⁺ (6)

When alkali is added, the pH may increase and the equilibrium may be driven to the right. As the vapour pressure of free SO₂ (e.g., H₂SO₃) is much higher than the vapour pressure of combined SO₂ (e.g., NaHSO₃), providing a lower pH may facilitate the collection and/or recovery of more SO₂ (e.g., by flashing).

Accordingly, by limiting the amount of alkali present in the pretreatment, which may result in a lower pH value, the percentage of SO₂ that may be readily recovered may increase. In addition, the percentage of SO₂ that may be trapped as combined SO₂ (e.g., NaHSO₃), and thus remain in the spent pretreatment liquor, is reduced. Recovering combined SO₂ (e.g., bisulfite salts in the spent pretreatment liquor) is more challenging and/or complex than recovering free SO₂, which may be freed using a pressure reduction or temperature increase.

In addition, by limiting the amount of alkali present in the pretreatment and by providing a sufficiently high concentration of SO₂, more lignosulfonic acid can be produced than alkali present (e.g., there may be more moles of sulfonated groups on the lignin than moles of alkali on a monovalent basis). This may further improve recovery of SO₂.

As SO₂ is driven out of solution (e.g., by flashing or evaporation), the pH of the solution may increase, which drives the equilibrium in Equation (6) to the right. However, by producing more lignosulfonic acid than alkali present, the pH of the solution may remain low as the SO₂ is driven off, such that the equilibrium is shifted to the left and such that less SO₂ is present as bisulfite salt.

In addition, limiting the amount of alkali present may improve pretreatment. In particular, the pH drop resulting from the formation of lignosulfonic acid may promote xylan dissolution, which may improve enzymatic hydrolysis.

Although low pH values have previously been associated with excessive acid-catalyzed hydrolysis of hemicellulose and/or cellulose, and/or with the formation of an excessive amount of potential fermentation inhibitors (e.g., furfural and HMF), it has been found that good glucose yields and reasonable xylose yields may be achieved using pretreatments at low pH (e.g., below 1.3) when the SO₂ loading is relatively high. Advantageously, these good results can be obtained without having to add an organic solvent (e.g., ethanol).

The acid bisulfite pretreatment may be carried out in batch mode, semi-batch mode, or continuous mode, in one or more pretreatment reactors. For example, the pretreatment may be conducted in one or more vertical reactors, horizontal reactors, inclined reactors, or any combination thereof. In one embodiment, the acid bisulfite pretreatment is carried out in batch mode in a steam autoclave. In one embodiment, the acid bisulfite pretreatment is conducted in continuous mode in a plug flow reactor. In one embodiment, the acid bisulfite pretreatment is conducted in a counter-current flow reactor. In one embodiment the acid bisulfite pretreatment is conducted in reactor provided with a charge of SO₂ as described in PCT Application No. PCT/CA2016/051089. In one embodiment, the acid bisulfite pretreatment is conducted in a digester (e.g., as commonly used in sulfite pulping).

In one embodiment, the acid bisulfite pretreatment is conducted in a pretreatment system, which may include a plurality of components/devices in addition to a pretreatment rector. Some examples of these devices/components include a biomass conveyer, washing system, dewatering system, a plug formation device, a heating chamber, a high shear heating chamber, a pre-steaming chamber, an SO₂ impregnation chamber, vapour reservoir chamber, an additional pretreatment reactor, connecting conduits, valves, pumps, etc.

In one embodiment, the acid bisulfite pretreatment is conducted in a pretreatment system and/or reactor that is pressurizable. For example, in one embodiment, the pretreatment reactor and/or pretreatment system includes a plurality of valves and/or other pressure increasing, pressure decreasing, or pressure maintaining components for providing and/or maintaining the pretreatment reactor at a specific pressure.

In general, the acid bisulfite pretreatment is conducted in a pretreatment system and/or pretreatment reactor that includes a heater, or some other heating means, for heating the lignocellulosic biomass to the pretreatment temperature. For example, in one embodiment, the pretreatment reactor is clad in a heating jacket. In another embodiment, the pretreatment reactor and/or the pretreatment system includes direct steam injection inlets (e.g., from a low pressure boiler). In one embodiment, the acid bisulfite pretreatment includes adding steam to provide a total pressure between about 190 psia and about 630 psia, between about 200 psia and about 600 psia, between about 250 psia and about 550 psia, or between about 300 psia and about 500 psia. For example, in one embodiment, where the total pressure is about 190 psia, the partial pressure of SO₂ may be about 21 psia, whereas the steam partial pressure may be about 169 psia.

At the end of the acid bisulfite pretreatment, the pretreated lignocellulosic biomass may be discharged from the pretreatment reactor and/or system. In one embodiment, this includes reducing the pressure on the pretreated lignocellulosic biomass. In general, the pressure may be released slowly or quickly. Alternatively, the pressure may be reduced at a stage further downstream. In one embodiment, the pressure is reduced by flashing.

### Preparing the Pretreated Biomass for Enzymatic Hydrolysis

In general, the pretreated material may be subject to one or more optional steps to prepare it for enzymatic hydrolysis. For example, in one embodiment the pretreated material is subject to a pressure reduction (e.g., flashing), a liquid/solid separation (e.g., filtering), a washing step, a cooling step, and/or a pH adjustment step.

In one embodiment, the pretreated biomass is subject to a pressure reduction. For example, in one embodiment, the pressure is reduced using one or more flash tanks in fluid connection with the pretreatment reactor. Flashing may reduce the temperature of the pretreated biomass to about 100° C. if an atmospheric flash tank, or lower if a vacuum flash tank.

In one embodiment, the pretreated biomass is subject to a liquid/solid separation, which provides a solid fraction and a liquid fraction. The solid fraction may contain undissolved solids such as unconverted cellulose and/or insoluble lignin. The liquid fraction, which may also be referred to as the xylose-rich fraction, may contain soluble compounds such as sugars (e.g., xylose, mannose, glucose, and arabinose), organic acids (e.g., acetic acid and glucuronic acid), soluble lignin (e.g., lignosulfonates), soluble sugar degradation products (e.g., furfural, which may be derived from C5 sugars, and HMF, which may be derived from C6 sugars) and/or one or more salts (e.g., sulfite salts). For example, in one embodiment, the pretreated biomass is flashed and then fed to one or more centrifuges that provide a solid stream and a liquid stream.

In one embodiment, the pretreated biomass is subject to one or more washing steps. For example, in one embodiment, the solid fraction from a solid/liquid separation is washed to remove soluble components, including potential inhibitors and/or inactivators. Washing may also remove soluble lignin (e.g., sulfonated lignin). In one embodiment, the pretreated biomass is washed as part of the liquid/solid separation (e.g., as part of decanter/wash cycle). The pretreated biomass may be washed as part of the liquid/solid separation at high or low pressure, which may or may not reduce the temperature of the pretreated biomass. In one embodiment, the wash water is reused or recycled. In one embodiment, the wash water and the liquid fraction are fed to fermentation. In one embodiment, lignin and/or lignosulfonates is extracted from the wash water.

In one embodiment, the pretreated biomass is subjected to one or more cooling steps. For example, in one embodiment, the pretreated biomass is cooled to within a temperature range compatible with enzyme(s) added for the enzymatic hydrolysis. For example, conventional cellulases often have an optimum temperature range between about 40° C. and about 65° C., and more commonly between about 50° C. and 65° C., whereas thermostable and/thermophilic enzymes may have optimum temperatures that are much higher (e.g., as high as, or greater than 80° C.). In one embodiment, the pretreated biomass is cooled to within a temperature range compatible with enzyme(s) and yeast used in a simultaneous saccharification and fermentation (SSF).

In general, the one or more cooling steps may include passive and/or active cooling of the liquid fraction, the solid fraction, or a combination of the liquids and solids. In one embodiment, the one or more cooling steps include flashing, heat exchange, washing, etc. In one embodiment, cooling is provided by injecting a fluid into the pretreated biomass. For example, in one embodiment, cooling is achieved when alkali and/or water is added to the pretreated biomass into order to provide the pH and/or consistency desired for enzymatic hydrolysis.

Advantageously, since the pretreatment is conducted at relatively low temperatures (e.g., between 120° C. and 150° C.), the one or more cooling steps may not have to produce a significant temperature drop.

In one embodiment, the pretreated biomass is subjected to one or more pH adjustment steps. In one embodiment, the pH of the pretreated biomass is adjusted to within a range near the pH optimum of the enzyme(s) used in hydrolysis. For example, cellulases typically have an optimum pH range between about 4 and about 7, more commonly between about 4.5 and about 5.5, and often about 5. In one embodiment, the pH is adjusted to between about 4 and about 8. In one embodiment, the pH is adjusted to between about 4.5 and about 6. In one embodiment, the pH is adjusted so as to substantially neutralize the pretreated biomass.

In one embodiment, the pH of the pretreated biomass is increased as a result of the washing step. In one embodiment, the pH of the pretreated biomass is increased by adding alkali (e.g., calcium hydroxide, potassium hydroxide, sodium hydroxide, ammonia gas, etc.). For example, in one embodiment, sufficient alkali is added to increase the pH of the pretreated biomass to a pH near the optimum pH range of the enzyme(s) used in hydrolysis. In one embodiment, the pH adjustment step includes adding sufficient alkali to overshoot the optimum pH of the enzyme (e.g., overliming), and then adding acid to reduce the pH to near the optimum pH range of the enzyme(s).

In general, the pH adjustment step may be conducted on the solid fraction, the liquid fraction, and/or a combination thereof, and may be conducted before, after, and/or as part of the one or more cooling steps. For example, in embodiments wherein the pretreated biomass is separated into a solid fraction and a liquid fraction, where only the solid fraction is fed to enzymatic hydrolysis, the pH of the liquid fraction may require adjustment prior to being fed to fermentation (e.g., separate from, or with, the hydrolyzate from the solid fraction). For example, in one embodiment, the pH of the liquid fraction is adjusted to the pH optimum of the microorganism used in a subsequent fermentation step. For example, Saccharomyces cerevisiae may have optimum pH values between about 4 and about 5.5.

Advantageously, since the pretreatment uses a relatively high amount of free SO₂ that is not combined with a cation, flashing of the pretreated biomass may remove a large portion of the SO₂, and thus increase the pH of the mixture, so that the pH adjustment step(s) may not have to significantly increase the pH and/or may require less alkali.

In one embodiment, enzyme is added to and/or mixed with the pretreated biomass (e.g., either the solid fraction or whole) prior to feeding the pretreated biomass to the hydrolysis reactor. In one embodiment, enzyme addition is after cooling and alkali addition.

As discussed above, the pretreated biomass may be washed. However, it can also be fed to enzymatic hydrolysis with minimal washing, or without washing. While washing may remove potential inhibitors and/or inactivators, and thus increase enzyme efficiency, it may also remove fermentable sugars, and thus reduce ethanol yield. Providing little or no washing of the pretreated biomass is advantageous in that it requires less process water and provides a simpler process.

Enzymatic Hydrolysis

The cellulose in the pretreated biomass can be hydrolyzed to glucose after enzyme addition. In one embodiment, enzyme addition includes the addition of cellulase. Cellulases are enzymes that can break cellulose chains into glucose. The term “cellulase”, as used herein, includes mixtures or complexes of enzymes that act serially or synergistically to decompose cellulosic material, each of which may be produced by fungi, bacteria, or protozoans. For example, in one embodiment, the cellulase is an enzyme cocktail comprising exo-cellobiohydrolases (CBH), endoglucanases (EG), and/or β-glucosidases (pG), which can be produced by a number of plants and microorganisms. Among the most widely studied, characterized and commercially produced cellulases are those obtained from fungi of the genera Aspergillus, Humicola, Chrysosporium, Melanocarpus, Myceliopthora, Sporotrichum and Trichoderma, and from the bacteria of the genera Bacillus and Thermobifida. Cellulase produced by the filamentous fungi Trichoderma longibrachiatum comprises at least two cellobiohydrolase enzymes termed CBHI and CBHII and at least four EG enzymes. As well, EGI, EGII, EGIlI, EGV and EGVI cellulases have been isolated from Humicola insolens. In addition to CBH, EG and PG, the cellulase may include several accessory enzymes that may aid in the enzymatic digestion of cellulose, including glycoside hydrolase 61 (GH61), swollenin, expansin, lucinen, and cellulose-induced protein (Cip). In one embodiment, the enzyme includes a lytic polysaccharide monooxygenase (LPMO) enzyme. For example, in one embodiment, the enzyme includes GH61. In one embodiment, the cellulase is a commercial cellulase composition that is suitable for use in the methods/processes described herein. In one embodiment, the cellulase includes CTec3, from Novozymes. In one embodiment, one or more cofactors are added. In one embodiment, O₂ or H₂O₂ is added. In one embodiment, ascorbic acid or some other reducing agent is added.

In one embodiment, enzyme addition is achieved by adding enzyme to a reservoir, such as a tank, to form an enzyme solution, which is then introduced to the pretreated biomass. In one embodiment, enzyme is added to the washed solid fraction of the pretreated biomass. In one embodiment, enzyme is added to a pH adjusted pretreated slurry that includes both liquid and solid portions of the pretreated biomass.

In general, the enzyme dose may depend on the activity of the enzyme at the selected pH and temperature, the reaction time, and/or other parameters. It should be appreciated that these parameters may be adjusted as desired by one of skill in the art.

In one embodiment, cellulase is added at a dosage between about 1 to 20 mg protein per gram cellulose (mg/g), at a dosage between about 2 to 20 mg protein per gram cellulose, at a dosage between about 1 to 15 mg protein per gram cellulose, or at a dosage between about 1 to 10 mg protein per gram cellulose. The protein may be quantified using either the bicinchoninic acid (BCA) assay or the Bradford assay.

In one embodiment, the initial concentration of cellulose in the slurry, prior to the start of enzymatic hydrolysis, is between about 0.01% (w/w) to about 20% (w/w). In one embodiment, the slurry fed to enzymatic hydrolysis is at a solids content between about 10% and 25%.

In one embodiment, the enzymatic hydrolysis is carried out at a pH and temperature that is at or near the optimum for the added enzyme. In one embodiment, the enzymatic hydrolysis is carried out at one or more temperatures between about 30° C. and about 95° C., between about 45° C. and about 65° C., between about 45° C. and about 55° C., or between about 50° C. and about 65° C. In one embodiment, the enzymatic hydrolysis is carried such that the pH value during the hydrolysis is between about 3.5 and about 8.0, between about 4 and about 6, or between about 4.8 and about 5.5.

In one embodiment, the enzymatic hydrolysis is carried out for a time between about 10 and about 250 hours, or between about 50 and about 250 hours. In one embodiment, the enzymatic hydrolysis is carried out for at least 24 hours, at least 36 hours, at least 48 hours, at least 72 hours, or at least 80 hours. In general, conducting the enzymatic hydrolysis for at least 24 hours may promote hydrolysis of both the amorphous and crystalline cellulose.

In one embodiment, the enzymatic hydrolysis includes agitation. Agitation may improve mass and/or heat transfer and may provide a more homogeneous enzyme distribution. In addition, agitation may entrain air in the slurry, which may be advantageous when the enzyme contains LPMO. In one embodiment, air and/or oxygen is added to the hydrolysis. In one embodiment, air and/or oxygen is added to the hydrolysis using a pump or compressor in order to maintain the dissolved oxygen concentration within a range that is sufficient for the full activity of LPMOs or any other oxygen-dependent components of the catalyst used to effect hydrolysis. In one embodiment, air or oxygen is bubbled into the slurry or headspace of one or more of the hydrolysis reactors.

In general, the enzymatic hydrolysis may be conducted as a batch process, a continuous process, or a combination thereof. In addition, the enzymatic hydrolysis may be agitated, unmixed, or a combination thereof. In one embodiment, the enzymatic hydrolysis is conducted in one or more dedicated hydrolysis reactors, connected in series or parallel. In one embodiment, the one or more hydrolysis reactors are jacketed with steam, hot water, or other heat sources.

In one embodiment, the enzymatic hydrolysis is conducted in one or more continuous stirred tank reactors (CSTRs) and/or one or more plug flow reactors (PFRs). In plug flow reactors, the slurry is pumped through a pipe or tube such that it exhibits a relatively uniform velocity profile across the diameter of the pipe/tube and such that residence time within the reactor provides the desired conversion. In one embodiment, the hydrolysis includes a plurality of hydrolysis rectors including a PFR and a CSTR in series.

In one embodiment, the enzymatic hydrolysis and fermentation are conducted in separate vessels so that each biological reaction can occur at its respective optimal temperature. In one embodiment, the enzymatic hydrolysis and fermentation are conducted is a same vessel, or series of vessels.

In one embodiment, the hydrolyzate provided by enzymatic hydrolysis is filtered to remove insoluble lignin and/or undigested cellulose.

### Fermentation

In one embodiment, the sugars produced during enzymatic hydrolysis and/or pretreatment are fermented via one or more microorganisms. In general, the fermentation microorganism(s) may include any suitable yeast and/or bacteria.

In one embodiment, at least a portion of the hydrolyzate produced during enzymatic hydrolysis is subjected to a fermentation with Saccharomyces spp. yeast. For example, in one embodiment, the fermentation is carried out with Saccharomyces cerevisiae, which has the ability to utilize a wide range of hexoses such as glucose, fructose, sucrose, galactose, maltose, and maltotriose to produce a high yield of ethanol. In one embodiment, the glucose and/or other hexoses derived from the cellulose are fermented to ethanol using a wild-type Saccharomyces cerevisiae or a genetically modified yeast. In one embodiment, the fermentation is carried out with Zymomonas mobilis bacteria.

In one embodiment, at least a portion of the hydrolyzate produced during enzymatic hydrolysis is fermented by one or more microorganisms to produce a fermentation broth containing butanol. For example, in one embodiment the glucose produced during enzymatic hydrolysis is fermented to butanol with Clostridium acetobutylicum.

In one embodiment, one or more of the pentoses produced during the pretreatment is fermented to ethanol using one or more organisms. For example, in one embodiment, xylose and/or arabinose produced during the pretreatment is fermented to ethanol with a yeast strain that naturally contains, or has been engineered to contain, the ability to ferment these sugars to ethanol. Examples of microbes that have been genetically modified to ferment xylose include recombinant Saccharomyces strains into which has been inserted either (a) the xylose reductase (XR) and xylitol dehydrogenase (XDH) genes from Pichia stipites.

In one embodiment, the xylose and other pentose sugars produced during the pretreatment are fermented to xylitol by yeast strains selected from the group consisting of Candida, Pichia, Pachysolen, Hansenula, Debaryomyces, Kluyveromyces and Saccharomyces.

In general, the C6 sugar from the enzymatic hydrolysis and the C5 sugar from the liquid fraction of the pretreated biomass can be subjected to separate fermentations or a combined fermentation. For example, consider the case where the pretreated biomass is subject to a solid/liquid separation and only the solid fraction is fed to enzymatic hydrolysis. In this case, the glucose produced by enzymatic hydrolysis can be fermented on its own, or can be combined with the liquid fraction and then fermented. For example, in one embodiment, a sugar solution containing both the C5 and C6 sugars is fermented to ethanol using only Saccharomyces cerevisiae. In one embodiment, a sugar solution containing both C5 and C6 sugars is fermented to ethanol using a mixture wherein C5 utilizing and ethanol producing yeasts (e.g., such as Pichia fermentans and Pichia stipitis) are cocultured with Saccharomyces cerevisiae. In one embodiment, a sugar solution containing both C5 and C6 sugars is fermented using genetically engineered Saccharomyces yeast capable of cofermenting glucose and xylose.

In general, the dose of the microorganism(s) will depend on a number of factors, including the activity of the microorganism, the desired reaction time, and/or other parameters. It should be appreciated that these parameters may be adjusted as desired by one of skill in the art to achieve optimal conditions. In one embodiment, the fermentation is supplemented with additional nutrients required for the growth of the fermentation microorganism. For example, yeast extract, specific amino acids, phosphate, nitrogen sources, salts, trace elements and vitamins may be added to the hydrolyzate slurry to support their growth. In one embodiment, yeast recycle is employed.

In one embodiment, the fermentation is carried out at a pH and temperature that is at or near the optimum for the added microorganism. For example, Saccharomyces cerevisiae may have optimum pH values between about 4 and about 5.5 and a temperature optimum between about 25° C. and about 35° C. In one embodiment, the fermentation is carried out at one or more temperatures between about 25° C. to about 55° C. In one embodiment, the fermentation is carried out at one or more temperatures between about 30° C. to about 35° C.

In general, the fermentation may be conducted as a batch process, a continuous process, or a fed-batch mode. For example, in one embodiment, the fermentation is conducted in continuous mode, which may offer greater productivity and lower costs. In one embodiment, the enzymatic hydrolysis may be conducted in one or more fermentation tanks, which can be connected in series or parallel. In general, the fermentation may be agitated, unmixed, or a combination thereof. For example, in one embodiment, the fermentation is conducted one or more continuous stirred tank reactors (CSTRs) and/or one or more plug flow reactors (PFRs). In one embodiment, the one or more fermentation tanks are jacketed with steam, hot water, or other heat sources.

In one embodiment, the enzymatic hydrolysis and fermentation are conducted in separate vessels so that each biological reaction can occur at its respective optimal temperature. In another embodiment, the hydrolysis (e.g., which may be also referred to as saccharification) is conducted simultaneously with the fermentation in same vessel. For example, in one embodiment, a simultaneous saccharification and fermentation (SSF) is conducted at temperature between about 35° C. and 38° C., which is a compromise between the 50° C. to 55° C. optimum for cellulase and the 25° C. to 35° C. optimum for yeast.

### Alcohol Recovery

Any alcohol produced during fermentation can be recovered, a process wherein the alcohol is concentrated and/or purified from the fermented solution (e.g., which may or may not have been subjected to a solids-liquid separation to remove unconverted cellulose, insoluble lignin, and/or other undissolved substances).

For example, in one embodiment, the fermentation produces ethanol, which is recovered using one or more distillation columns that separate the ethanol from other components (e.g., water). In general, the distillation column(s) in the distillation unit may be operated in continuous or batch mode, although are typically operated in a continuous mode. Heat for the distillation process may be introduced at one or more points, either by direct steam injection or indirectly via heat exchangers. After distillation, the water remaining in the concentrated ethanol stream (i.e., vapour) may be removed from the ethanol rich vapour by a molecular sieve resin, by membrane extraction, or other methods known to those of skill in the art for concentration of ethanol beyond the 95% that is typically achieved by distillation (e.g., a vapour phase drying). The vapour may then be condensed and denatured.

### Sulfur Dioxide Recovery

Excess SO₂ not consumed during the pretreatment can be recovered and/or recycled. For example, in one embodiment, SO₂ not consumed during the pretreatment is forced out of the pretreated slurry by a pressure reduction (e.g., top relief, atmospheric flash, vacuum flash, vacuum, etc.) or by a temperature increase (e.g., evaporation by heating). The SO₂ forced out of the pretreated slurry can be collected, recovered, and/or recycled within the process. In one embodiment, the SO₂ forced out of the pretreated slurry is fed to an SO₂ recovery unit. For example, in one embodiment, the slurry of pretreated material is flashed, and the flash stream, which contains the excess SO₂, is fed to a SO₂ recovery unit.

In general, the SO₂ recovery unit may be based on any suitable SO₂ recovery technology, as known in the art. In one embodiment, the SO₂ recovery unit includes a partial condenser, an SO₂ stripper, and/or an SO₂ scrubbing system. In one embodiment, the SO₂ recovery unit includes a SO₂ scrubbing system, which may include one or more packed absorbers (e.g., amine-based, alkali-based, or other absorbers). In one embodiment, the SO₂ recovery unit provides purified SO₂ that can be recycled for use in the pretreatment. In one embodiment, the SO₂ recovery unit provides partially purified SO₂ that can be recycled for use in the pretreatment.

In one embodiment, the recovered SO₂, which is optionally stored temporarily, is recycled directly back into the process. Advantageously, SO₂ recovery allows the recycling of sulfur within the system, and thus improves the process economics (e.g., since less SO₂ needs to be acquired for pretreatment).

As described herein, the SO₂ recovery is improved by limiting the amount of alkali present during the pretreatment to a concentration less than about 0.42 wt % (OH, on liquor), while increasing the amount of SO₂ to a level that provides an initial pH less than 1.3.

### Lignosulfonate Recovery

In one embodiment, lignosulfonate generated during the pretreatment is recovered. The term lignosulfonate refers to water soluble sulfonated lignin (i.e., soluble in water at neutral and/or acid conditions) and encompasses both lignosulfonic acid and its neutral salts. In general, lignosulfonate may be recovered following pretreatment, enzymatic hydrolysis, and/or fermentation. In one embodiment, lignosulfonate is recovered for energy production (e.g., combusted). In one embodiment, lignosulfonate is recovered for producing value-added materials (e.g., a dispersing agent, a binding agent, a surfactant, an additive in oil and gas drilling, an emulsion stabilizer, an extrusion aid, to produce vanillin, for dust control applications, etc.).

In general, lignosulfonate may be recovered by any method used to produce lignosulfonate products (e.g., provided in liquid form or as a powder). For example, lignosulfonate may be recovered using a method conventionally used for recovering lignosulfonates from waste liquor (e.g., brown or red) of a sulfite pulping process. In one embodiment, lignosulfonate is recovered by precipitation and subsequent filtering, membrane separation, amine extraction, ion exchange, dialysis, or any combination thereof.

To facilitate a better understanding of embodiments of the instant invention, the following examples are given. In no way should the following examples be read to limit, or define, the entire scope of the invention.

### EXAMPLES

### Example 1: Acid Bisulfite Pretreatment of Lignocellulosic Material

Acid bisulfite pretreatment of sugar cane bagasse was conducted in 25 mL, stainless steel, laboratory tubular reactors (i.e., about 5 inches in length). The bagasse had a cellulose/glucan content of 40.13%, xylan content of 22.26%, a lignin content of 25.40%, and a total solids (TS) content of 91.66%, w/w on a dry basis. The carbohydrate assay was based on Determination of Structural Carbohydrates and Lignin in Biomass-LAP (Technical Report NRELITP-510-42618).

Stock sulfurous acid solution having a SO₂ concentration between about 11.7 wt % and about 12.5 wt % (on liquor) (e.g., about 15 wt % to 16 wt % H₂SO₃ on liquor) was prepared by bubbling SO₂ into Milli-Q water cooling in an ice bath. The exact concentration of the sulfurous acid stock solution was determined using back titration with HCl (0.1M). The sulfurous acid stock solution was stored at about 4° C. Stock NaHSO₃ solutions were prepared by adding NaHSO₃ to degassed Milli-Q water and stored in filled sealed vials to eliminate headspace.

Pretreatment slurries were prepared by adding bagasse to each laboratory tubular reactor, followed by stock NaHSO₃ solution, and a quantity of water calculated to provide the target SO₂ and alkali concentrations (e.g., based on the concentration of the stock sulfurous acid solution). Once the cooled stock sulfurous acid solution was added to this mixture, the reactors were sealed immediately. Each reactor was cooked at the pretreatment temperature of 140° C., in an oil bath, for the selected pretreatment time. The pretreatment time shown includes the time for the reactor to reach the pretreatment temperature (e.g., about 5 minutes).

At the end of the pretreatment, the reactors were cooled in an ice bath. All experiments conducted with or based on SO₂/sulfurous acid were carried out in a fume hood.

The concentrations used and conditions for the acid bisulfite pretreatment are summarized in Table 1.

TABLE 1
Pretreatment conditions

| | |
|---|---|
| Concentration of SO₂ (wt %, on liquor) | 10.5 |
| Concentration of H₂SO₃ (wt %, on liquor) | 13.5 |
| Solids consistency (wt %) | 10 |
| Concentration of SO₂ (wt %, on dry weight of bagasse) | 94.5 |
| Concentration of H₂SO₃ (wt %, on dry weight of bagasse) | 121.4 |
| Concentration of NaHSO₃ (g/L) | 10 |
| NaHSO₃ loading (wt %, on dry weight of bagasse) | 9.0% |
| Concentration of alkali (from NaHSO₃) (wt %, OH, on liquor) | 0.16 |
| Concentration of alkali (from NaHSO₃) (wt %, OH, on dry | 1.47 |
| weight of bagasse) | |
| Pretreatment temperature (° C.) | 140 |
| Pretreatment time (min) | 180 |
| Initial pH | 0.92-0.99 |
| Final pH | 0.63-0.7 |

The pH of the cooled slurry of pretreated bagasse (e.g., at ambient temperature) was 0.63. This acid bisulfite pretreatment provided a xylose yield of 50.41 (wt % based on potential xylose available in the feedstock) and a residual xylan of 2.21 (wt %, based on xylan initially present). This acid bisulfite pretreatment solubilized 73.37% of the lignin (wt %, based on lignin initially present).

The carbohydrate content of the pretreated material can be ascertained with a carbohydrate assay based on Determination of Structural Carbohydrates and Lignin in Biomass-LAP (Technical Report NREL/TP-510-42618). This assay can provide the cellulose content, xylan content, insoluble lignin content, and soluble lignin content of the pretreated biomass, w/w on a dry basis. The residual xylan and lignin solubilization/dissolution are calculated relative to the untreated lignocellulosic biomass. The concentration of monomeric sugars (e.g., glucose and/or xylose) and the corresponding yields may be determined using high performance liquid chromatography (HPLC). For the results described herein, the cellulose/glucan content, xylan content, lignin content, xylose yield, etc. were determined using the methodology set out in the Examples in U.S. Pat. No. 9,574,212.

### Example 2: Enzymatic Hydrolysis

Washed pretreatment samples were prepared by suspending a portion of pretreated sample in ultra-purified water (Milli-Q™), filtering the suspension through glass fiber filter paper (G6, 1.6 microns), and then repeating the alternating steps.

The washed pretreatment solids were hydrolyzed in 50 mL Erlenmeyer flasks, at a consistency of 15 wt %, with sodium citrate (1 M of citrate buffer pH added to a final concentration of 0.1M). The flasks were incubated at 52° C., with moderate shaking at about 250 rpm, for 30 minutes to equilibrate substrate temperature.

Hydrolysis was initiated by adding liquid cellulase enzyme. Enzyme was added at a dosage of 2.5-9 mg/g (i.e., mg protein/g of cellulose). The flasks were incubated at 52° C. in an orbital shaker (250 rpm) for various hydrolysis times (e.g., 200 hours).

The hydrolyses were followed by measuring the sugar monomers in the hydrolysate. More specifically, aliquots obtained at various hours of hydrolysis, were used to analyze the sugar content. More specifically, HPLC was used to measure the amount of glucose, which was used to determine the cellulose conversion. The cellulose conversion, which is expressed as the amount of glucose released during enzymatic hydrolysis of the solid fraction, and thus sometimes is referred to as glucose conversion, was determined using the following equation and the methodology outlined in Example 9 of U.S. Pat. No. 9,574,212.

Cellulose conversion=concentration of glucose in aliquot/maximum glucose concentration at 100% conversion.

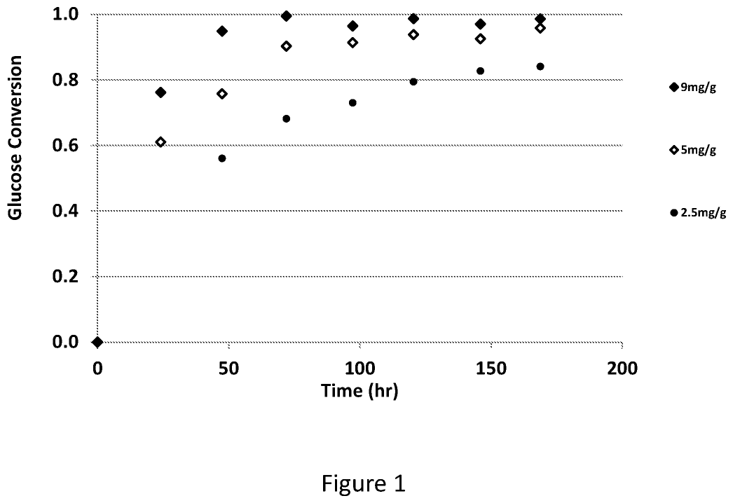

FIG. 1 shows a plot of glucose conversion for the washed solids of the acid bisulfite pretreatment summarized in Table 1, for enzyme loadings of 2.5 mg/g, 5 mg/g, and 9 mg/g. Remarkably, the acid bisulfite pretreatment permitted more than 80% glucose conversion for the enzymatic hydrolysis for all three enzyme doses, including the low dose of 2.5 mg/g. Enzyme doses of 5 mg/g and 9 mg/g were able to provide a glucose conversion of more than 90%.

The glucose conversions shown in FIG. 1 demonstrate that acid bisulfite pretreatment can permit good enzymatic hydrolyses even when the concentration of alkali is limited to less than about 0.2 wt % (OH, on liquor). For example, assuming that the bagasse has an inherent alkali concentration of about 0.2 wt % (on solids), which corresponds to about 0.02 wt % (on liquor), the concentration of alkali in this system would be about 0.18 wt % (on liquor). Although the pretreatment used a relatively high SO₂ concentration (e.g., greater than about 10 wt % (on liquor), the SO₂ recovery and/or processing of the spent pretreatment liquor is expected to be improved as a result of the relatively low alkali concentration. Surprisingly, the acid bisulfite pretreatment solubilized 73.37% of the lignin (wt %, based on lignin initially present). This is surprising because low pH values in pretreatment are generally associated with a relatively high residual lignin content and/or because high bisulfite salt concentrations are believed to be required in lignin solubilization. Solubilizing lignin may be advantageous in terms of improving enzymatic hydrolysis and/or providing an increased lignosulfonate yield.

Of course, the above embodiments have been provided as examples only. It will be appreciated by those of ordinary skill in the art that various modifications, alternate configurations, and/or equivalents will be employed without departing from the spirit and scope of the invention. Accordingly, the scope of the invention is therefore intended to be limited solely by the scope of the appended claims.

1. A process for processing lignocellulosic biomass comprising:
(i) pretreating lignocellulosic biomass, said pretreating comprising heating the lignocellulosic biomass in a pretreatment liquor containing sulfur dioxide and bisulfite salt, said heating conducted between 120° C. and 150° C., for at least 30 minutes, wherein initially a pH of the pretreatment liquor at 25° C. is less than 1.3, a concentration of sulfur dioxide is greater than 9.4 wt % (on liquor), and a concentration of alkali is between 0 wt % and 0.42 wt % (expressed as hydroxide, on liquor);
(ii) obtaining a slurry of pretreated lignocellulosic biomass produced in (i), said slurry having a solid fraction comprising cellulose and a liquid fraction comprising solubilized hemicellulose;
(iii) forcing sulfur dioxide out of the liquid fraction, wherein said liquid fraction has a pH at 25° C. that is less than 1;
(iv) enzymatically hydrolyzing at least a portion of the cellulose in the solid fraction to glucose;
(v) fermenting the glucose to an alcohol, and
(vi) recovering the alcohol.

2. The process according to claim 1, wherein a ratio of the concentration of sulfur dioxide to the concentration of alkali is greater than 20.

3. The process according to claim 1, wherein a ratio of the concentration of sulfur dioxide to the concentration of alkali is greater than 25.

4. The process according to claim 1, wherein the concentration of sulfur dioxide is between 9.4 wt % (on liquor) and 19.5 wt % (on liquor).

5. The process according to claim 1, wherein the pH of the pretreatment liquor at 25° C. is less than 1.1.

6. The process according to claim 1, wherein the pH of the pretreatment liquor at 25° C. is less than 1.

7. The process according to claim 1, wherein said heating is conducted between 125° C. and 145° C.

8. The process according to claim 1, comprising subjecting the slurry of pretreated lignocellulosic biomass produced in (i) or (ii) to a solids-liquid separation to separate the solid fraction and the liquid fraction.

9. The process according to claim 8, comprising washing the solid fraction produced by the solids-liquid separation with water, and adding cellulase to the washed solid fraction.

10. The process according to claim 1, wherein pretreating the lignocellulosic biomass comprises contacting the lignocellulosic biomass with the pretreatment liquor, and the pretreatment liquor contains sufficient alkali to provide the alkali concentration between 0 wt % and 0.42 wt % (expressed as hydroxide, on liquor).

11. The process according to claim 1, wherein the sulfite salt comprises at least one of sodium bisulfite, potassium bisulfite, or ammonium bisulfite.

12. The process according to claim 1, wherein the lignocellulosic biomass comprises a non-woody lignocellulosic biomass.

13. The process according to claim 1, wherein the lignocellulosic biomass comprises a woody lignocellulosic biomass.

14. The process according to claim 1, wherein the lignocellulosic biomass comprises wheat straw or bagasse.

15. The process according to claim 1, wherein forcing sulfur dioxide out of the liquid fraction comprises subjecting the slurry of pretreated lignocellulosic biomass to a pressure reduction.

16. The process according to claim 15, comprising collecting the sulfur dioxide forced out of the liquid fraction for recycle within the process.

17. The process according to claim 1, wherein the alcohol is ethanol.

18. The process according to claim 1, wherein the slurry of pretreated lignocellulosic biomass obtained in (ii) comprises sulfonated lignin, and wherein the concentration of alkali (on liquor) is less than a concentration of sulfonated groups on the sulfonated lignin (on liquor) in the slurry of pretreated lignocellulosic biomass.

19. The process according to claim 8, comprising producing one or more products from the liquid fraction, said one or more products comprising at least one of xylose, xylitol, methane, ethanol, or lignosulfonate.

20. A process for processing lignocellulosic biomass comprising:
(i) pretreating lignocellulosic biomass, said pretreating comprising heating the lignocellulosic biomass in a pretreatment liquor containing sulfur dioxide and bisulfite salt, said heating conducted between 110° C. and 150° C., for at least 30 minutes, wherein initially a pH of the pretreatment liquor at 25° C. is less than 1.3, a concentration of sulfur dioxide is greater than 36 wt % (on dry solids), and a concentration of alkali is less than 0.25 wt % (expressed as hydroxide, on liquor);
(ii) obtaining a slurry of pretreated lignocellulosic biomass produced in (i), said slurry having a solid fraction comprising cellulose and a liquid fraction comprising solubilized hemicellulose;
(iii) forcing sulfur dioxide out of the liquid fraction, wherein said liquid fraction has a pH at 25° C. that is less than 1;
(iv) enzymatically hydrolyzing at least a portion of the cellulose in the solid fraction to glucose;
(v) fermenting the glucose to an alcohol, and
(vi) recovering the alcohol.

21. A process for processing lignocellulosic biomass comprising:
(i) pretreating lignocellulosic biomass, said pretreating comprising heating the lignocellulosic biomass in a pretreatment liquor containing sulfur dioxide and bisulfite salt, said heating conducted between 110° C. and 150° C., for at least 30 minutes, wherein initially a pH of the pretreatment liquor at 25° C. is less than 1.3, and wherein a ratio of a concentration of sulfur dioxide on liquor to a concentration of alkali expressed as hydroxide, on liquor, is greater than 30;
(ii) obtaining a slurry of pretreated lignocellulosic biomass produced in (i), said slurry having a solid fraction comprising cellulose and a liquid fraction comprising solubilized hemicellulose;
(iii) forcing sulfur dioxide out of the liquid fraction, wherein said liquid fraction has a pH at 25° C. that is less than 1;
(iv) enzymatically hydrolyzing at least a portion of the cellulose in the solid fraction to glucose;
(v) fermenting the glucose to an alcohol, and
(vi) recovering the alcohol.
